(19) United States
(12) Patent Application Publication
(10) Pub. No.: US 2021/0392835 A1
(43) Pub. Date: Dec. 23, 2021

(54) A CONTROL SYSTEM

(71) Applicant: Provectus IP Pty Ltd, Queensland (AU)

(72) Inventor: Nusqe SPANTON, Queensland (AU)

(21) Appl. No.: 17/287,570

(22) PCT Filed: Oct. 22, 2019
(86) PCT No.: PCT/AU2019/051152

§ 371 Date: Apr. 22, 2021

(30) Foreign Application Priority Data
Oct. 22, 2018 (AU) 2018903996

(51) Int. Cl. A01G 33/00; A01G 9/24; A01G 7/02; H05B 45/10; H05B 45/20; H05B 47/175; H05B 47/105

(52) U.S. Cl. CPC A01G 33/00; A01G 9/249; A01G 7/02; H05B 47/105; H05B 45/20; H05B 47/175; H05B 45/10

(57) ABSTRACT

A control system for growing plant matter such as algae, the control system having a controller for controlling a plurality of LED lights, wherein the controller is adapted to control the spectrum and intensity of the LED lights. Preferably the controller is also adapted to control a flickering frequency of the LED lights.

### FIELD OF INVENTION

The present invention relates to a control system for growing algae. Reference will be made in the specification to the use of the invention with respect to growing algae. The patent specification describes this use, but it is by way of example only and the invention is not limited to this use.

### BACKGROUND OF THE INVENTION

Commercial cultivation of Algae is used to produce food ingredients, food colorants and dyes, bioplastics, pharmaceuticals, and algal fuel amongst others. Most Algae requires water, nutrients, a carbon source and light to grow.

How all these elements are administered determines the quality and quantity of the algae. There are a large number of combinations of how these elements can be administered to grow different types of algae.

Light is an important element in growing algae. Direct sunlight is too strong for most algae. However, direct sunlight is often best for strong growth as the algae underneath the surface is able to utilize the less intense light created from the shade above.

One prior art solution to control the exposure of algae to light is to agitate the algae.

### OBJECT OF THE INVENTION

It is an object of the present invention to overcome or at least alleviate one or more of the above mentioned problems with growing algae and/or provide the consumer with a useful or commercial choice.

### SUMMARY OF THE INVENTION

In one aspect, the present invention broadly resides in a control system for growing plant matter having

a controller for controlling a plurality of LED lights,

wherein the controller is adapted to control the spectrum and intensity of the LED lights.

Preferably, the controller is adapted to control a flickering frequency of the LED lights. Preferably the flickering frequency is a frequency of on/off cycles of the LED light. Preferably the controller is adapted to control the flickering frequency in a range of 2 ms to 5,000 ms. More preferably the controller is adapted to control the flickering frequency in a range of 25 ms to 1,000 ms. In another embodiment, the controller is adapted to control the flickering frequency in a range of 2 ms to 500 ms. In a further embodiment, the controller is adapted to control the flickering frequency in a range of 10 ms to 100 ms.

Preferably the controller includes a communication module. The communication module is preferably adapted to communicate with a remote device. In one embodiment, the remote device is a mobile device such as a mobile telephone. In another embodiment, the remote device is a computer. In a further embodiment, the remote device is a server. Preferably the controller communicates with the server via the internet.

In one embodiment, the controller is adapted to communicate with more than one remote device.

The remote device is preferably adapted to set the spectrum and intensity of the LED lights. The remote device is preferably adapted to set the flickering frequency of the LED lights.

Preferably the controller is adapted to communicate with one or more sensors. Preferably the one or more sensors provide data on the growth of the plant matter.

Preferably the one or more sensors include one or more gas sensors. Preferably the one or more gas sensors include a carbon dioxide, hydrogen and/or oxygen sensor. In one embodiment, the one or more gas sensors are adapted to sense dissolved gas in growing medium used to grow plant matter such as algae. The growing medium is preferably a liquid such as water.

Preferably the one or more sensors include a sensor to determine plant growth. Preferably the one or more sensors includes a spectrophotometer to determine plant growth. In one embodiment, the one or more sensors includes a spectrophotometer to determine algae cell density in liquid culture.

Preferably the one or more sensors include a temperature sensor. Preferably the temperature sensor senses a temperature of the growing medium.

Preferably the one or more sensors includes a pH sensor to measure the pH of the growing medium.

Preferably the one or more sensors include a salinity sensor. Preferably the salinity sensor measures the salinity of the growing medium.

Preferably the one or more sensors include a nitrate sensor. Preferably the nitrate sensor measures the concentration of nitrates in the growing medium.

Preferably the one or more sensors include a cell count sensor. Preferably the cell count sensor measures the quantity of plant matter cells in a defined volume.

Preferably the one or more sensors include a cell health sensor. Preferably the cell health sensor provides data on the health of plant matter cells.

The controller is preferably adapted to receive data from the one or more sensors. The controller is preferably adapted to send the data from the one or more sensors to the remote device. The controller is preferably adapted to send data on the spectrum and intensity of the LED lights to the remote device. The controller is preferably adapted to send data on the flickering frequency of the LED lights to the remote device. The remote device is preferably adapted to optimise the spectrum and intensity of the LED lights based on the data from the one or more sensors. Preferably the remote device correlates data from the one or more sensors with data on the spectrum and intensity of the LED lights. Preferably the remote device correlates data from the one or more sensors with data on the flickering frequency of the LED lights. Preferably the remote device will determine the spectrum and intensity of the LED lights that correspond to desirable data from the one or more sensors. Preferably the remote device will set the spectrum and intensity values in the controller to the determined spectrum and intensity of the LED lights. Preferably the remote device will determine the flickering frequency of the LED lights that corresponds to desirable data from the one or more sensors. Preferably the remote device will set the flickering frequency in the controller to the determined flickering frequency of the LED lights.

Preferably the remote device will correlate the type of plant matter being grown with the data on the spectrum and intensity of the LED lights. Preferably the remote device will correlate the type of plant matter being grown with the data from the one or more sensors. Preferably the remote device will correlate the type of plant matter being grown with the data on the flickering frequency of the LED lights.

In one embodiment, a remote device defines the type of plant matter being cultivated and communicates this data with the controller, the controller communicates this data with a further remote device and downloads from the further remote device spectrum and intensity settings of the LED lights which correlate with the type of plant matter being cultivated.

Preferably the remote device will correlate desired traits of plant matter being grown with the data on the spectrum and intensity of the LED lights. Preferably the remote device will correlate desired traits of plant matter being grown with the data from the one or more sensors. Preferably the remote device will correlate desired traits of plant matter being grown with the data on the flickering frequency of the LED lights.

In one embodiment, a remote device defines the desired traits of plant matter being cultivated and communicates this data with the controller, the controller communicates this data with a further remote device and downloads from the further remote device spectrum and intensity settings of the LED lights which correlate with the desired traits of plant matter being cultivated.

In another embodiment, the remote device is used to manually set the spectrum and intensity settings of the LED lights in the controller.

Preferably the controller is adapted to control the spectrum of the LED lights in the range of 200 nm to 800 nm. More preferably the controller is adapted to control the spectrum of the LED lights in the range of 380 nm to 750 nm.

Preferably the LED lights to provide the plant matter with light.

Preferably the controller is adapted to control each of the plurality of LED lights individually. In one embodiment the plurality of LED lights are divided into zones, with the controller adapted to control each zone individually. In one embodiment, each zone is used to cultivate different plant matter. In another embodiment, each zone is used to cultivate plant matter at a different growth stage. In a further embodiment, each zone is used to develop different traits in the plant matter.

Preferably the plant matter is algae.

Preferably the control system includes the LED lights.

Preferably the control system includes the one or more sensors.

In one embodiment, the control system includes the remote device.

In another aspect, the present invention broadly resides in an algae cultivation control system, the control system having

a controller for controlling a plurality of LED lights to provide the algae with light,

wherein the controller is adapted to control the spectrum and intensity of the LED lights.

In a further aspect, the present invention broadly resides in a control system for growing algae having

a controller for controlling a plurality of LED lights,

wherein the controller is adapted to control the spectrum and intensity of the LED lights.

In another aspect, the present invention broadly resides in in a control system for growing algae having

a plurality of LED lights to provide the algae with light; and

a controller for controlling the plurality of LED lights,

wherein the controller is adapted to control the spectrum and intensity of the LED lights.

Preferably the control system includes one or more sensors. Preferably the one or more sensors provide data on the growth of the algae.

Preferably the control system includes a communication module. More preferably, the controller includes the communication module. The communication module is preferably adapted to communicate with a remote device. The communication module is preferably adapted to enable communication between the remote device and the controller.

In one embodiment, the remote device is adapted to communicate with one or more further control systems. Preferably the remote device is adapted to communicate the settings for the spectrum and intensity of the LED lights which have resulted in desirable traits in the plant matter to the one or more further control systems.

Preferably the remote device is adapted to communicate additional settings to the control system and or the one or more further control systems. Preferably the additional settings include one or more of flow rate of growing medium, temperature of growing medium, amount of CO₂ added, amount of O₂ added, and or the type and or amount of nutrients added. Preferably the corresponding controller is adapted to control one or more pumps, valves, solenoids, heaters, or the like, to control the flow rate of growing medium, temperature of growing medium, amount of CO₂ added, amount of O₂ added, and or the type and or amount of nutrients added. Preferably the corresponding controller controls one or more pumps, valves, solenoids, heaters, or the like, to control the flow rate of growing medium, temperature of growing medium, amount of CO₂ added, amount of O₂ added, and or the type and or amount of nutrients added, according to the settings received from the remote device.

In a further aspect, the present invention broadly resides in a method of controlling the growing of algae, the method including the steps of

controlling with a first control system the spectrum and intensity of a plurality of LED lights for growing algae;

monitoring with one or more sensors traits of the algae;

changing the spectrum and intensity of the plurality of LED lights while continuing to monitor with the one or more sensors traits of the algae;

correlating traits of the algae with the spectrum and intensity of the plurality of LED lights;

transmitting with a remote device the spectrum and intensity settings of the plurality of LED lights for a desired trait of the algae to a second control system to grow algae with the desired traits using the second control system.

Preferably the method includes the step of transmitting from the first control system to the remote device the spectrum and intensity settings of the plurality of LED lights and the correlating sensor data for the traits of the algae. Preferably the step of correlating traits of the algae with the spectrum and intensity of the plurality of LED lights is performed on the remote device.

Preferably the method further includes correlating traits of the algae with the flow rate of growing medium, temperature of growing medium, amount of CO₂ added, amount of O₂ added, and or the type and or amount of nutrients added. Preferably the method includes changing one or more of the spectrum and or intensity settings of the plurality of LED lights, flow rate of growing medium, temperature of growing medium, amount of CO₂ added, amount of O₂ added, and or the type and or amount of nutrients added to optimise the traits of the algae. Preferably Al is used to change one or more of the spectrum and or intensity settings of the plurality of LED lights, flow rate of growing medium, temperature of growing medium, amount of CO₂ added, amount of O₂ added, and or the type and or amount of nutrients added to optimise the traits of the algae. Preferably the method further includes transmitting with the remote device settings related to one or more of flow rate of growing medium, temperature of growing medium, amount of CO₂ added, amount of O₂ added, and or the type and or amount of nutrients added to the second control system to grow algae with the desired traits using the second control system.

In another aspect, the present invention broadly resides in a method of controlling the growing of algae, the method including the steps of

controlling with a first control system the spectrum and intensity of a plurality of LED lights for growing algae;

monitoring with one or more sensors traits of the algae;

changing the spectrum and intensity of the plurality of LED lights while continuing to monitor with the one or more sensors traits of the algae;

correlating traits of the algae with the spectrum and intensity of the plurality of LED lights;

controlling the spectrum and intensity settings of the plurality of LED lights to correspond to a desired trait of the algae.

It will be appreciated that traits of the algae can include cell count, growth rate, cell health, algae cell density and or the like.

The features described with respect to one aspect also apply where applicable to all other aspects of the invention. Furthermore, different combinations of described features are herein described and claimed even when not expressly stated. For example the features described in relation to the control system for growing plant matter can apply to the algae cultivation control system and the control system for growing algae and vice versa.

### BRIEF DESCRIPTION OF THE DRAWINGS

In order that the present invention can be more readily understood reference will now be made to the accompanying drawings which illustrate a preferred embodiment of the invention and wherein:

FIG. 1 is a schematic view of a control system according to an embodiment of the present invention;

FIG. 2 is a schematic view of a portion of a control system according to another embodiment of the present invention; and

FIG. 3 is a schematic view of a portion of a control system according to a further embodiment of the present invention.

### DETAILED DESCRIPTION OF THE PREFERRED EMBODIMENT

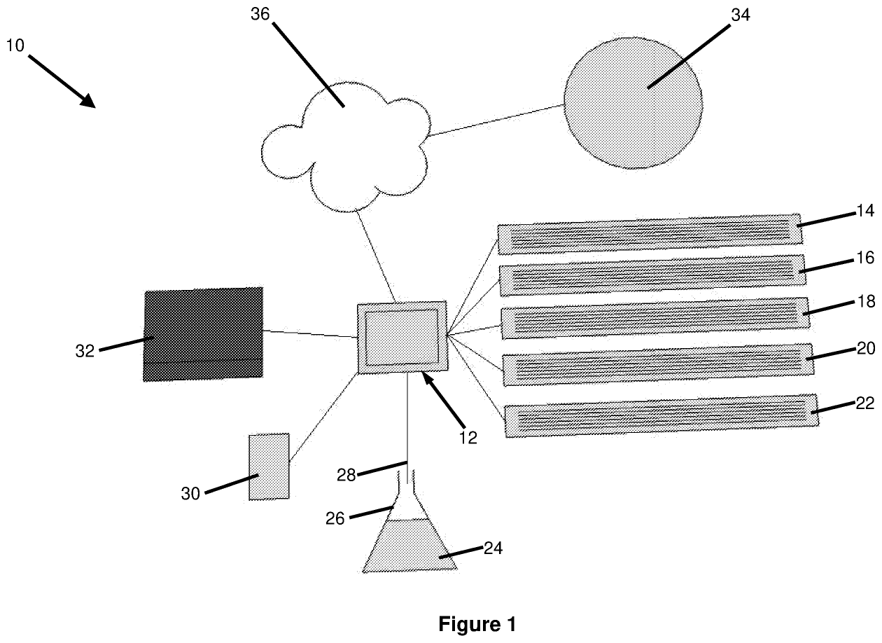

With reference to FIG. 1 there is shown a control system according to an embodiment of the present invention in the form of an algae cultivation control system 10. The control system 10 includes a controller 12.

The controller 12 controls a plurality of LED lights 14,16,18,20,22. The controller 12 is adapted to control the spectrum of the LED lights 14,16,18,20,22. The controller 12 is adapted to control the intensity of the LED lights 14,16,18,20,22.

The controller 12 is adapted to control the flickering frequency of the LED lights 14,16,18,20,22.

The LED lights 14,16,18,20,22 provide light to plant matter in the form of algae 24 being grown/cultivated, in a growing medium in the form of water, in a container 26.

One or more sensors 28 monitor the growth of the algae and communicate the data to the controller 12.

The controller 12 communicates with remote devices in the form of a mobile device 30, a computer 32 and a server 34. The controller 12 communicates with the server 34 via the internet 36.

The controller 12 is able to communicate the settings of the LED lights 14,16,18,20,22 and the data from the one or more sensors 28 to the mobile device 30, computer 32 and server 34. The settings of the LED lights 14,16,18,20,22 in the controller 12 can be set by the mobile device 30, the computer 32 and/or the server 34.

The server 34 can correlate data from the one or more sensors 28 with settings of the spectrum, the intensity and/or the flickering frequency of the LED lights 14,16,18,20,22. The server 34 can determine the spectrum, the intensity and/or the flickering frequency of the LED lights 14,16,18,20,22 that correspond to desirable data from the one or more sensors 28. The server 34 can set the spectrum, intensity and/or the flickering frequency values in the controller 12 to the determined values. The mobile device 30, the computer 32 and/or the server 34 can set which data from the one or more sensors 28 is desirable.

The server 34 can correlate data for different types of algae being grown and/or different traits of the algae 24 being grown. The mobile device 30, the computer 32 and/or the server 34 can set the different types of algae being grown, or the traits of the algae being grown.

The controller 12 can control the spectrum, the intensity and/or the flickering frequency of the LED lights 14,16,18,20,22 individually. In this manner, LED lights 14,16,18,20,22 can define different zones, in which different algae is being grown and/or in which different traits of algae are being cultivated.

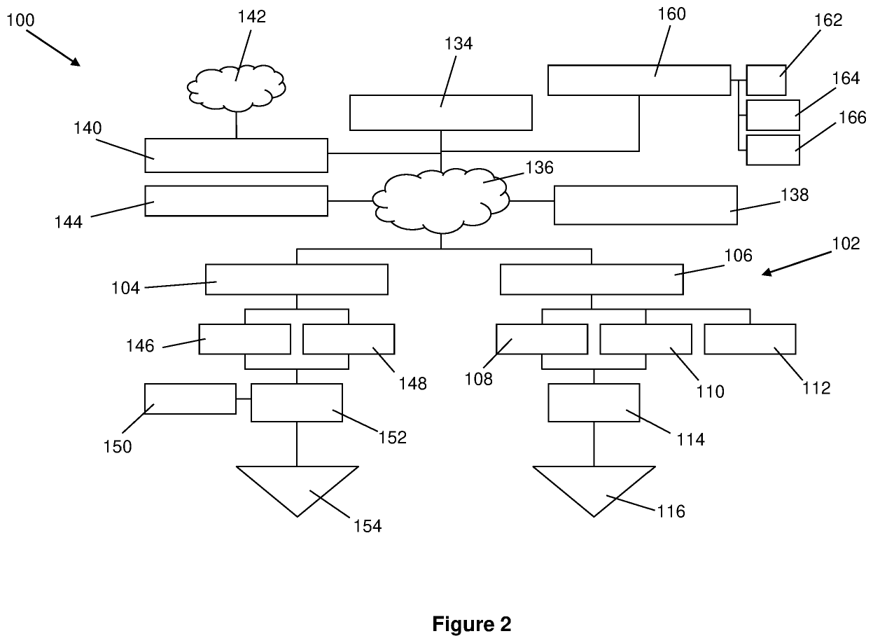

With reference to FIG. 2, there is shown an algae cultivation control system 100. The algae cultivation control system 100 includes a first control system in the form of a pilot control system 102, and a second control system in the form of a user control system 104.

The pilot control system 102 includes a controller 106 for controlling the spectrum, intensity and flickering rate settings of the plurality of LED lights, flow rate of growing medium, temperature of growing medium, amount of CO₂ added, amount of O₂ added, and or the type and or amount of nutrients added in the cultivation of algae at 108 and 110. The controller 106 also monitors sensors (not shown) which provide data on the growth of the algae at 108 and 110. The controller 106 also monitors the waste products created at 112. At 114 the grown algae is processed and at 116 the end product is dispatched. The pilot plant is used to test the effects of different settings of the spectrum, intensity and flickering rate settings of the plurality of LED lights, flow rate of growing medium, temperature of growing medium, amount of CO₂ added, amount of O₂ added, and or the type and or amount of nutrients added in the cultivation of algae at 108 and 110.

The data from the sensors and the settings of the spectrum, intensity and flickering rate settings of the plurality of LED lights, flow rate of growing medium, temperature of growing medium, amount of CO₂ added, amount of O₂ added, and or the type and or amount of nutrients added is sent via the internet 136 to a remote device in the form of a server 134.

The server 134 processes the data and settings in an optimisation module 140. The optimisation module 140 uses artificial intelligence 142 to optimise the settings to achieve desired traits of the algae. The server 134 maintains a database of optimised settings for different algae strains and different desired traits. On request through a pilot user console 138, the server 134 can communicate the updated settings to the pilot control system 102.

If a user is growing a known strain of algae using the user control system 104, they can via a user console 144, download optimised settings from the server 134 to the user control system 104, to control the cultivation of algae at 146 and 148. The user control system also monitors the waste production at 150. At 152 the grown algae is processed and at 154 the end product is dispatched.

Data and settings from the user control system 104 is uploaded via the internet 136 to the server 134. The data and settings are fed into the optimisation module 140 so that the artificial intelligence 142 can use the data and settings to further optimise the settings to achieve desired traits of the algae.

The server 134 includes a notification module 160 which can notify users and pilot users (not shown) via sms 162, email 164 or AV 166 to new algae recipes or optimisations, or alert users if settings or data are outside of a predetermined range for the pilot control system 102 or user control system 104 respectively.

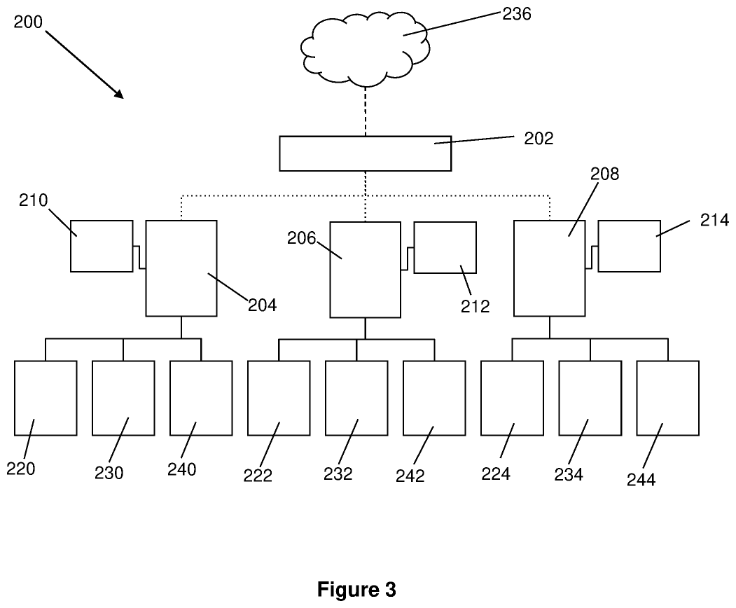

With reference to FIG. 3, there is shown a control system 200. The control system 200 has a controller 202 which controls multiple sections of an algae cultivation system in the form of an inoculation section 204, a grow section 206 and an inoculation section 208.

Each of the sections 204, 206, 208 has a localised controller 210,212,214. The localised controllers 210,212,214 each control LED lights 220,222,224 respectively. The localised controllers 210,212,214 each control solenoids and pumps 230,232,234 respectively to control the flow rate of growing medium, amount of CO₂ added, amount of O₂ added, and or the type and or amount of nutrients added. The localised controllers 210,212,214 also monitor sensors 240,242,244. The controller 202 can send setting and sensor data to a remote server (not shown) via the internet 236 via Internet of Things (IoT) messaging system. The controller can also receive IoT messages to control the LED lights 220,222,224 and the solenoids and pumps 230,232,234 via the respective localised controllers 210,212,214

### ADVANTAGES

An advantage of the preferred embodiment of the control system includes the ability to control the growth of different types of plant matter. Another advantage of the preferred embodiment of the control system includes the ability to control different traits of plant matter being grown. A further advantage of the preferred embodiment of the control system includes that the controller can set the spectrum, intensity and/or the flickering frequency values of the LED lights in response to desired data values from the one or more sensors.

### VARIATIONS

While the foregoing has been given by way of illustrative example of this invention, all such and other modifications and variations thereto as would be apparent to persons skilled in the art are deemed to fall within the broad scope and ambit of this invention as is herein set forth.

Throughout the description and claims of this specification the word “comprise” and variations of that word such as “comprises” and “comprising”, are not intended to exclude other additives, components, integers or steps.

1. A control system for growing plant matter, comprising:
a controller for controlling a plurality of LED lights, wherein the controller is adapted to control the spectrum and intensity of the LED lights, and
wherein the controller is adapted to control a flickering frequency of the LED lights in a range of 2 ms to 5,000 ms.

2. A control system as claimed in claim 1, wherein the controller is adapted to control the flickering frequency in a range of 10 ms to 100 ms.

3. A control system as claimed in claim 1, wherein the controller includes a communication module adapted to communicate with a remote device.

4. A control system as claimed in claim 3, wherein the remote device is adapted to set a spectrum and an intensity of the LED lights.

5. A control system as claimed in claim 4, further comprising one or more sensors to provide data on the growth of the plant matter,
wherein the controller is adapted to receive data from the one or more sensors and send the data from the one or more sensors to the remote device, and the remote device is adapted to optimise the spectrum and intensity of the LED lights based on the data from the one or more sensors.

6. A control system as claimed in claim 5, wherein the controller is adapted to send data on the flickering frequency of the LED lights to the remote device, and the remote device determines the flickering frequency of the LED lights that corresponds to desirable data from the one or more sensors, and the remote device will set the flickering frequency of the LED lights to the determined flickering frequency of the LED lights.

7. A control system as claimed in claim 1, wherein the plant matter is algae.

8. An algae cultivation control system, the control system comprising a controller for controlling a plurality of LED lights to provide the algae with light, wherein the controller is adapted to control a spectrum and an intensity of the LED lights, and wherein the controller is adapted to control a flickering frequency of the LED lights in a range of 2 ms to 5,000 ms.

9. A control system for growing algae having a controller for controlling a plurality of LED lights, wherein the controller is adapted to control the spectrum and intensity of the LED lights, and wherein the controller is adapted to control a flickering frequency of the LED lights in a range of 2 ms to 5,000 ms.

10. A control system for growing algae comprising:
a plurality of LED lights to provide the algae with light; and
a controller for controlling the plurality of LED lights, wherein the controller is adapted to control the spectrum and intensity of the LED lights, and wherein the controller is adapted to control a flickering frequency of the LED lights in a range of 2 ms to 5,000 ms.

11. A control system as claimed in claim 9, further comprising
one or more sensors to provide data on the growth of the algae; and
a communication module adapted to communicate sensor data and spectrum and intensity settings of the LED lights to a remote device.

12. A control system as claimed in claim 11, wherein the remote device is adapted to communicate the settings for the spectrum and intensity of the LED lights which have resulted in desirable traits in the algae to one or more further control systems.

13. A control system as claimed in claim 11, wherein the remote device is adapted to communicate additional settings including one or more of flow rate of growing medium, temperature of growing medium, amount of CO2 added, amount of O2 added, and or the type and or amount of nutrients added, to the control system and or the one or more further control systems, and wherein the corresponding controller is adapted to control one or more pumps, valves, solenoids, heaters, or the like, to control the flow rate of growing medium, temperature of growing medium, amount of CO2 added, amount of O2 added, and or the type and or amount of nutrients added.

14. A method of controlling the growing of algae, the method comprising the steps of:
controlling with a first control system a spectrum and an intensity of a plurality of LED lights for growing algae;
controlling with the first control system a flickering frequency of the plurality of LED lights in a range of 2 ms to 5,000 ms,
monitoring with one or more sensors traits of the algae;
changing the spectrum, intensity and flickering frequency of the plurality of LED lights while continuing to monitor with the one or more sensors traits of the algae;
correlating traits of the algae with the spectrum, intensity and flickering frequency of the plurality of LED lights; and
transmitting with a remote device the spectrum, intensity and flickering frequency settings of the plurality of LED lights for a desired trait of the algae to a second control system to grow algae with the desired traits using the second control system.

15. A method as claimed in claim 14, further comprising the step of transmitting from the first control system to the remote device the spectrum, intensity and flickering frequency settings of the plurality of LED lights and the correlating sensor data for the traits of the algae, and
wherein the step of correlating traits of the algae with the spectrum, intensity and flickering frequency of the plurality of LED lights is performed on the remote device.

16. A method as claimed in claim 14, further comprising the step of correlating traits of the algae with a flow rate of growing medium, temperature of growing medium, amount of CO2 added, amount of O2 added, and or the type and or amount of nutrients added.

17. A method as claimed in claim 16 further comprising the step of transmitting with the remote device settings related to one or more of the flow rate of growing medium, temperature of growing medium, amount of CO2 added, amount of O2 added, and or the type and or amount of nutrients added to the second control system to grow algae with the desired traits using the second control system.

18. A method of controlling the growing of algae, the method comprising the steps of:
controlling with a control system a spectrum and an intensity of a plurality of LED lights for growing algae;
controlling with the control system a flickering frequency of the plurality of LED lights in a range of 2 ms to 5,000 ms,
monitoring with one or more sensors traits of the algae;
changing the spectrum, intensity and flickering frequency of the plurality of LED lights while continuing to monitor with the one or more sensors traits of the algae;
correlating traits of the algae with the spectrum. intensity and flickering frequency of the plurality of LED lights; and
controlling the spectrum, intensity and flickering frequency settings of the plurality of LED lights to correspond to a desired trait of the algae.
